(19) United States
(12) Patent Application Publication
(10) Pub. No.: US 2026/0294241 A1
(43) Pub. Date: Oct. 1, 2026

(54) SLIT LAMP SYSTEM AND METHOD OF SYNCHRONIZING IMAGE ACQUISITION USING ELECTRONIC DEVICES THEREOF

(71) Applicant: LIGHTX INNOVATIONS INC., Québec (CA)

(72) Inventor: Nicolas DÉPELTEAU, Québec (CA)

(21) Appl. No.: 19/631,503

(22) Filed: Mar. 27, 2026

Related U.S. Application Data
(60) Provisional application No. 63/780,765, filed on Mar. 31, 2025.

(51) Int. Cl. A61B 3/135

(52) U.S. Cl. CPC A61B 3/135

(57) ABSTRACT

The slit lamp system generally has: a slit lamp apparatus; a first electronic device having a first camera, a second electronic device having a second camera, the first and second electronic devices communicating with one another via a communication link having a maximal latency value; and an image acquisition actuator transmitting an image acquisition instruction to the first electronic device upon actuation; wherein, upon receiving the image acquisition instruction, the first electronic device simultaneously: instructing the first camera to acquire a first image at a given moment in time subsequent to said receiving the image acquisition instruction by the maximal latency value; and transmitting a given instruction signal to the second electronic device via the communication link, the given instruction signal instructing the second camera to acquire a second image at the given moment in time.

### FIELD

The improvements generally relate to eye examination and more specifically relates to eye examination using binocular-type slit lamps.

### BACKGROUND

A binocular-type slit lamp is an instrument having an illumination source assembly that can shine a thin strip of light into the eye of a patient, and a binocular microscope for observing the illuminated eye through two ocular elements for examination purposes. Slit lamps are generally operated by optometrists, ophthalmologist and other eye care professionals as they typically require a high level of training to suitably illuminate some specific parts of the eye in precise conditions and assess the condition of the illuminated eye. In some circumstances, capturing images of the eye of a patient in a stereoscopic manner can enable a perception of depth in the resulting stereoscopic image which can help the eye care professionals in the eye examination. To do so, it was known to acquire the images using two cameras each facing a respective ocular of the binocular microscope. Although existing slit lamp systems were satisfactory to a certain degree, there remained room for improvement.

### SUMMARY

For instance, it was found that manually actuating the two cameras could result in a considerable mistiming which could introduce undesirable artifacts in the resulting stereoscopic image. Moreover, it was also found that by arranging the two cameras in a conventional leader-follower configuration with which the two cameras can be actuated electronically could still lead to stereoscopic images of subpar quality at least in some circumstances. Indeed, it was found that such typical leader-follower configurations do not consider an instantaneous load on a communication link established between the two cameras, and even more so when the cameras are part of a corresponding electronic device which may accomplish a plurality of simultaneous and independent tasks at any time. Accordingly, there was a need in the industry to solve at least some of the aforementioned drawbacks. More specifically, this disclosure relates to synchronizing the image acquisition of two cameras in a way which factors in an instantaneous latency existing in a communication link established between the two electronic devices, and which may be direct or indirect.

In accordance with a first aspect of the present disclosure, there is provided a slit lamp system comprising: a slit lamp apparatus having a patient area; a first electronic device having a first camera facing the patient area, a second electronic device having a second camera facing the patient area, the first and second electronic devices communicating with one another via a communication link having a maximal latency value Δtmax; and an image acquisition actuator communicatively coupled to the first electronic device and transmitting an image acquisition instruction to the first electronic device upon actuation; wherein, upon receiving the image acquisition instruction, the first electronic device performs the steps of: instructing the first camera to acquire a first image at a given moment in time t1 subsequent to said receiving the image acquisition instruction by the maximal latency value Δtmax; and transmitting a given instruction signal to the second electronic device via the communication link, the given instruction signal instructing the second camera to acquire a second image at the given moment in time t1, resulting in the first image and the second image being acquired simultaneously.

Further in accordance with the first aspect of the present disclosure, the first electronic device is configured for determining the maximal latency value Δtmax, said determining the maximal latency value Δtmax including: i) the first electronic device transmitting a test signal to the second electronic device, ii) the first electronic device receiving a return of the test signal from the second electronic device, iii) measuring a time delay between said transmitting the test signal and said receiving the return, and iv) dividing the time delay by two to yield an instantaneous latency value Δti.

Still further in accordance with the first aspect of the present disclosure, the first and second electronic devices are configured for determining the maximal latency value Δtmax, said determining the maximal latency value Δtmax including: i) the first electronic device transmitting a test signal to the second electronic device at a first timestamp, ii) the second electronic device receiving the test signal from the first electronic device at a second timestamp, iii) measuring a time delay between the second timestamp and the first timestamp, and associating the time delay to an instantaneous latency value Δti.

Still further in accordance with the first aspect of the present disclosure, the slit lamp system further comprising determining a plurality of instantaneous latency values Δti over time, the maximal latency value Δtmax corresponding to an upper limit of the plurality of instantaneous latency values Δti.

Still further in accordance with the first aspect of the present disclosure, said determining the plurality of instantaneous latency values over time is performed once every 500 ms, and preferably once every 250 ms.

Still further in accordance with the first aspect of the present disclosure, the maximal latency value Δtmax corresponds to a maximum of a number of immediately preceding latency values Δti, the number of immediately preceding latency values Δti including at most 20 immediately preceding latency values Δti, preferably at most 10 immediately preceding latency values Δti, and most preferably at most 5 immediately preceding latency values Δti.

Still further in accordance with the first aspect of the present disclosure, the first electronic device is one of: a smart phone, an electronic table, and a digital single-lens reflex (DSLR) camera system.

Still further in accordance with the first aspect of the present disclosure, the image acquisition actuator is a physical actuator.

Still further in accordance with the first aspect of the present disclosure, the image acquisition actuator is a graphical element of a graphical user interface.

Still further in accordance with the first aspect of the present disclosure, the graphical user interface is part of any one of the first and second electronic devices.

Still further in accordance with the first aspect of the present disclosure, the slit lamp apparatus has a mounting bracket mounted thereto, the mounting bracket receiving the first and second electronic devices.

In accordance with a second aspect of the present disclosure, there is provided a method of synchronizing image acquisition using a first electronic device and a second electronic device, the first electronic device having a first camera, the second electronic device having a second camera, the method comprising: establishing a communication link between the first electronic device and the second electronic device; determining a maximal latency value Δtmax of the communication link, the maximal latency value Δtmax indicative of a maximal time delay required for a test signal to be communicated between the first electronic device and the second electronic device; upon receiving an image acquisition instruction at the first electronic device, the first electronic device instructing the first camera to acquire a first image at a given moment in time t1 subsequent to said receiving the image acquisition instruction by said maximal latency value Δtmax; and the first electronic device transmitting an instruction signal to the second electronic device via the communication link, the instruction signal instructing the second camera to acquire a second image at the given moment in time t1, resulting in the first image and the second image being acquired simultaneously.

Further in accordance with the second aspect of the present disclosure, said determining the maximal latency value Δtmax includes: i) transmitting the test signal to the second electronic device, ii) receiving a return of the test signal from the second electronic device, iii) measuring a given time delay between said transmitting the test signal and said receiving the return, and iv) dividing the time delay by two to yield an instantaneous latency value Δti.

Still further in accordance with the second aspect of the present disclosure, determining the maximal latency value Δtmax includes: i) the first electronic device transmitting the test signal to the second electronic device at a first timestamp, ii) the second electronic device receiving the test signal from the first electronic device at a second timestamp, iii) measuring a given time delay between the second timestamp and the first timestamp, and associating the time delay to an instantaneous latency value Δti.

Still further in accordance with the second aspect of the present disclosure, the method further comprising determining a plurality of instantaneous latency values Δti over time, the maximal latency value Δtmax corresponding to an upper limit of the plurality of instantaneous latency values Δti.

Still further in accordance with the second aspect of the present disclosure, said determining the plurality of instantaneous latency values Δti is performed once every 500 ms, and preferably once every 250 ms.

Still further in accordance with the second aspect of the present disclosure, the maximal latency value Δtmax corresponds to a maximum of a number of immediately preceding latency values Δti, the number of immediately preceding latency values Δti including at most 20 immediately preceding latency values Δti, preferably at most 10 immediately preceding latency values Δti, and most preferably at most 5 immediately preceding latency values Δti.

Still further in accordance with the second aspect of the present disclosure, the method further comprising determining a statistical distribution of the plurality of instantaneous latency values Δti, wherein the maximal latency value Δtmax corresponding to a median of the statistical distribution plus a variance value σ of the statistical distribution.

Still further in accordance with the second aspect of the present disclosure, the method can for example further comprise determining a statistical distribution of the plurality of instantaneous latency values Δti, the maximal latency value Δtmax corresponding to a 95^{th} percentile of the statistical distribution.

Still further in accordance with the second aspect of the present disclosure, the communication link is one of a Bluetooth® communication link, a Wi-Fi communication link, and a dedicated communication link.

Still further in accordance with the second aspect of the present disclosure, the test signal has a size matching an expected size of the instruction signal.

In accordance with a third aspect of the present disclosure, there is provided a slit lamp system comprising: a slit lamp apparatus having a patient area; a first electronic device having a first camera facing the patient area, a second electronic device having a second camera facing the patient area, the first and second electronic devices communicating with one another via a communication link; and an image acquisition actuator communicatively coupled to the first electronic device and transmitting an image acquisition instruction to the first electronic device upon actuation; wherein, upon receiving the image acquisition instruction, the first electronic device performs the steps of: instructing the first camera to acquire a first image at a given moment in time t1 subsequent to said receiving the image acquisition instruction by a given time value; and transmitting a given instruction signal to the second electronic device via the communication link, the given instruction signal instructing the second camera to acquire a second image at the given moment in time t1, resulting in the first image and the second image being acquired simultaneously.

Further in accordance with the third aspect of the present disclosure, the communication link can for example have a maximal latency value Δtmax, the given time corresponding to the maximal latency value Δtmax of the communication link.

All technical implementation details and advantages described with respect to a particular aspect of the present invention are self-evidently mutatis mutandis applicable for all other aspects of the present invention.

Many further features and combinations thereof concerning the present improvements will appear to those skilled in the art following a reading of the instant disclosure.

### DESCRIPTION OF THE FIGURES

In the figures,

FIG. 1 is an exploded view of an example of a slit lamp system having a slit lamp apparatus, a mounting bracket, a first electronic device, a second electronic device, and a controller, in accordance with one or more embodiments;

FIG. 2A is an example of a first image captured by a first camera of the first electronic device at a given moment in time, in accordance with one or more embodiments;

FIG. 2B is an example of a second image captured by a second camera of the second electronic device at the given moment in time, in accordance with one or more embodiments;

FIG. 3 is a stereoscopic image formed using the first image of FIG. 2A and the second image of FIG. 2B, in accordance with one or more embodiments;

FIG. 4 is a flow chart of an example of a method of synchronizing image acquisition using the first and second electronic devices of FIG. 1, in accordance with one or more embodiments;

FIG. 5A is a schematic view showing, at moment in time ta, the transmission of a test signal from a first electronic device to a second electronic device and, at a moment in time tb, the reception of the test signal at the second electronic device, in accordance with one or more embodiments;

FIG. 5B is a schematic view showing, at moment in time tc, the return of the test signal from the second electronic device to the first electronic device, in accordance with one or more embodiments;

FIG. 5C is a timeline showing exemplary latency values measured using timestamps associated to the moments in time ta, tb and tc of FIGS. 5A and 5B, in accordance with one or more embodiments;

FIG. 6 is a graph showing instantaneous latency values Δti measured over time, in accordance with one or more embodiments;

FIG. 7 is a graph showing a distribution of instantaneous latency values Δti, in accordance with one or more embodiments;

FIG. 8 is a schematic timeline showing steps performed by a first electronic device and a second electronic device during an example method of synchronizing image acquisition, in accordance with one or more embodiments; and

FIG. 9 is a schematic view of an example of a computing device of the controller of FIG. 1, in accordance with one or more embodiments.

### DETAILED DESCRIPTION

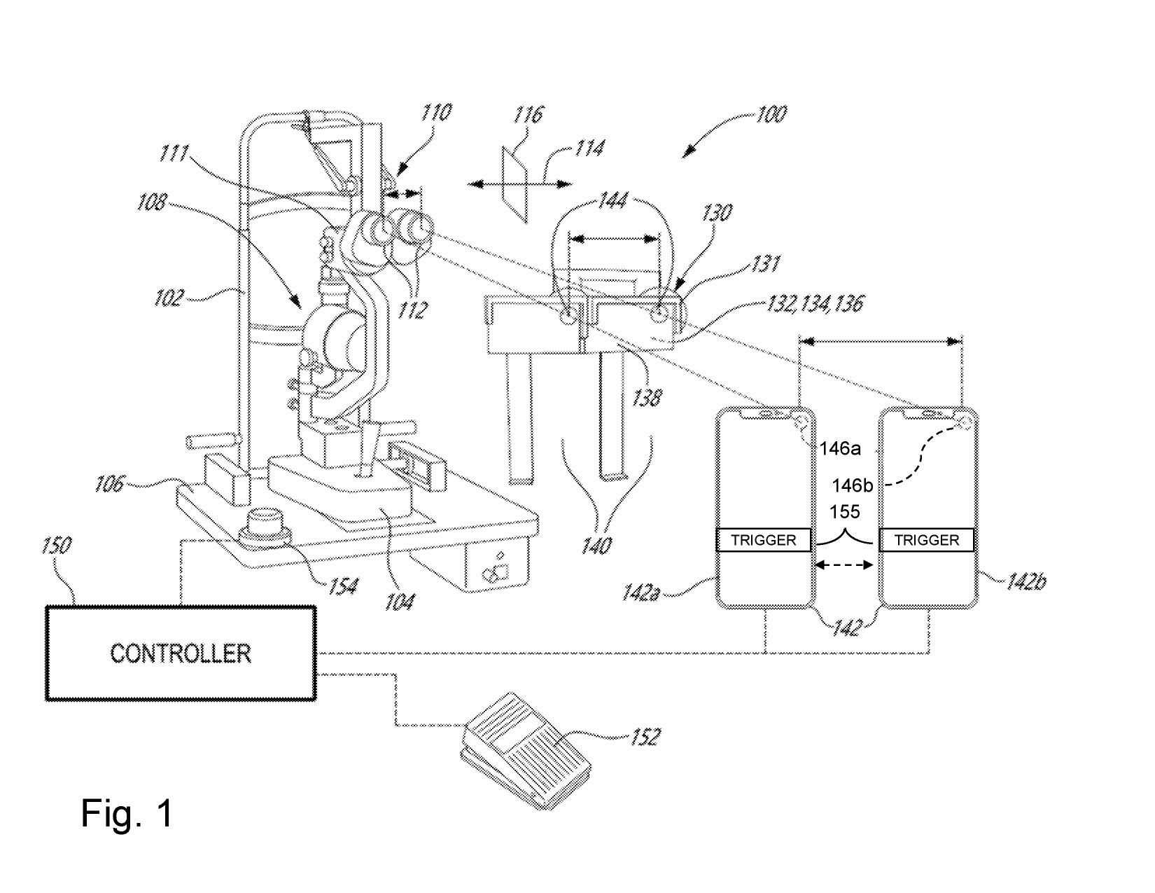

FIG. 1 shows an example of a slit lamp system 100 for imaging an eye of a patient, in accordance with a specific embodiment. As shown, the slit lamp system 100 has a frame 102 supporting components of the slit lamp system 100. The frame 102 generally has a base 104 resting on a support surface 106 such as a tabletop, a floor and the like.

As depicted, the slit lamp system 100 has an illumination source assembly 108 adapted to illuminate the eye of the patient in one or more illumination patterns. Examples of such illumination patterns can include, but are not limited to, diffuse illumination, direct focal illumination, tangential illumination, retroillumination, indirect illumination, sclerotic scatter illumination, and any combination thereof. The illumination source assembly 108 can be positioned above the head of the patient, such as in Haag Streit type slit lamps, or below the head of the patient, such as in Zeiss type slit lamps. As such, the illumination source assembly 108 can include a variety of other optical components, such as shutter(s), mirror(s), diffuser(s), filter(s) and the like to propagate, carry and/or modify the light generated by a slit illuminator and a background illuminator, for instance.

The slit lamp system 100 also has an imaging assembly 110 which is mounted to the frame 102. The imaging assembly 110 has a number of optical elements adapted to image the eye of the patient during illumination as provided by the illumination source assembly 108. As shown, the imaging assembly 110 is of the binocular type. More specifically, the binocular imaging assembly 110 has a binocular scope 111 optically coupled to two ocular elements 112 which are transversally spaced apart from one another. In this disclosure, the transverse orientation 114 is generally perpendicular to a sagittal plane 116 of the slit lamp system 100 and to the vertical orientation. As best shown in FIG. 1A, the binocular scope 111 and the two ocular elements 112 collectively form images of the eye E propagating along corresponding eye imaging paths, with each ocular elements forming a corresponding image at an imaging plane 118 axially spaced apart along an axis A of the ocular element 112 during the imaging process. As shown, the imaging assembly 110 can include a variety of other optical components, such as shutter(s), mirror(s), diffuser(s), filter(s) and the like to propagate, carry and/or modify the light incoming from the eye E of the patient. In conventional eye examination, a health care professional can view the images of the eye E by placing her/his eyes at the imaging planes 118 formed before the ocular elements 112.

Referring back to FIG. 1, the slit lamp system 100 has a mounting bracket 130 to be removably attached to the slit lamp system 100, and more specifically to the ocular elements 112 in this specific embodiment. The mounting bracket 130 has a body 131 with a first face 132 which has features 134 being directly or indirectly mounted to the ocular elements 112. More specifically, in this embodiment, the mounting bracket 130 has ocular mounting members 136 which are attachable, clipable or otherwise mountable on the ocular elements 112 of the binocular imaging assembly 110. In some other embodiments, the mounting bracket 130 may be directly or indirectly mounted with respect to the ocular elements 112. In other words, the mounting bracket 130 may be mounted directly to the frame, but place before the ocular elements 112. The mounting bracket 130 has a second face 138 opposite to the first face 132. As shown, the second face 138 has two transversally spaced-apart camera receivers 140 which are each configured to removably receive a corresponding one of a pair of cameras. As shown, the cameras can be part of corresponding electronic devices 142. As such, in some embodiments, the camera receivers 140 can be provided in the form of electronic device receivers. The mounting bracket 130 also has two transversally spaced-apart camera through apertures 144 extending between the first and second faces 132 and 138 of the mounting bracket 130.

In this embodiment, there are provided two electronic devices 142, namely a first electronic device 142a and a second electronic device 142b. Each of the electronic devices 142 may be standalone in the sense that they each include a processor, a computer-readably memory having instructions which when executed by the processor can perform predetermined steps. For instance, either or both of the electronic devices 142 may run a respective operating software with which software applications can be installed and run when desired. As depicted, each of the electronic devices 142 have a respective camera. More specifically, the first electronic device 142a has a first camera 146a whereas the second electronic device 142b has a second camera 146b. Generally, the cameras 146 typically have a resolution greater than 2 megapixels, preferably greater than 4 megapixels, and more preferably more than 8 megapixels. When the two electronic devices 142 are suitably received in the camera receivers 140 of the mounting bracket 130, the first and second cameras 146a and 146b of the electronic devices 142 face the patient. More specifically, the first and second cameras 146a and 146b are exposed to the imaging planes of the binocular imaging assembly 110 via the camera apertures 144. The type of electronic device can include, but is not limited to, a smartphone such as the iPhone® (any generation), the Android® phone (any generation) and the like, an electronic tablet such as the iPad® (any generation), the Android® tablet (any generation), a digital single-lens reflex (DSLR) camera system, any camera system, and the like. The electronic devices can be of a similar type in some embodiments, or of dissimilar types in some other embodiments.

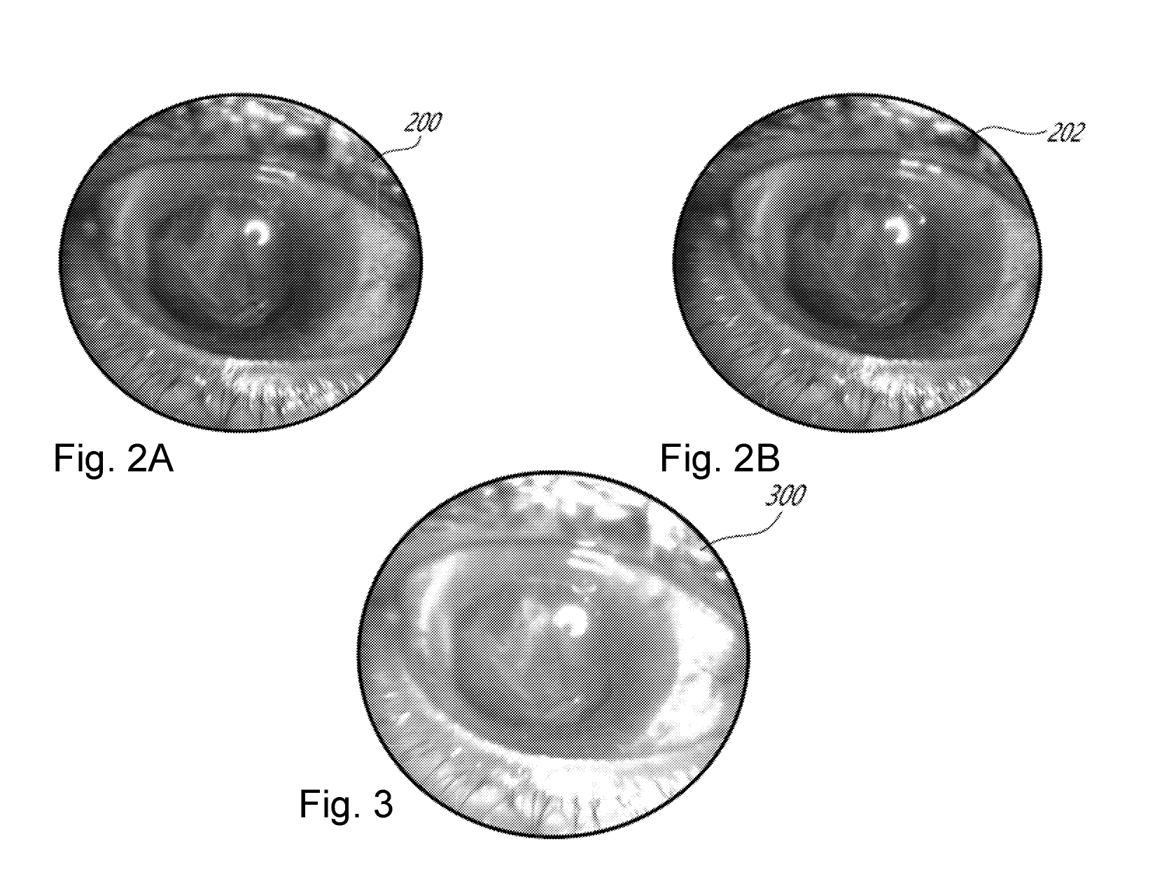

As such, the electronic devices 142 can simultaneously capture the images of the eye of the patient formed at the imaging planes of the two ocular elements 112, examples of which are shown in FIGS. 2A and 2B. More specifically, FIG. 2A shows a first image 200 captured by the first camera 146a whereas FIG. 2B shows a second image 202 captured by the second camera 146b. By associating the two simultaneously captured images to one another, a stereo image pair can be obtained thereby allowing three-dimensional perception of the eye of the patient. The stereo image pair can be processed to obtain a stereoscopic image 300, an example of which is shown in FIG. 3.

Referring back to FIG. 1, the slit lamp system 100 has a controller 150 which may be communicatively coupled to the first electronic device 142a, the second electronic device 142b, and/or a number of physical or virtual actuators. As shown in this specific embodiment, the slit lamp system 100 has a first physical actuator provided in the form of a foot pedal 152, and a second physical actuator provided in the form of a push button 154. As such, during use, an eye care professional may actuate either the foot pedal 152 and/or the push button 154 when a simultaneous image acquisition is desired, which may in turn cause the controller 150 to send an image acquisition instruction to either one or both of the first and second electronic devices 142a and 142b. The communication between the controller 150 and the first and second electronic devices 142a and 142b may be wired or wireless, or a combination of both, depending on the embodiment. In some other embodiments, display screens of the electronic device(s) 142 may display a graphical element acting as a virtual actuator. Accordingly, the eye care professional may actuate a physical actuator, such as the foot pedal 152 or the push button 154, or a graphical element 155 of a graphical user interface displayed on the electronic device(s) 142. Regardless of whether the image acquisition instruction originates from the physical actuators via the controller 150, or from the electronic devices 142 via the graphical element 155, the first and second electronic devices 142a and 142b are configured for performing a method of synchronizing image acquisition which can ensure that the first and second images are acquired at the same time, or quasi the same time, thereby yielding a stereoscopic image of higher quality.

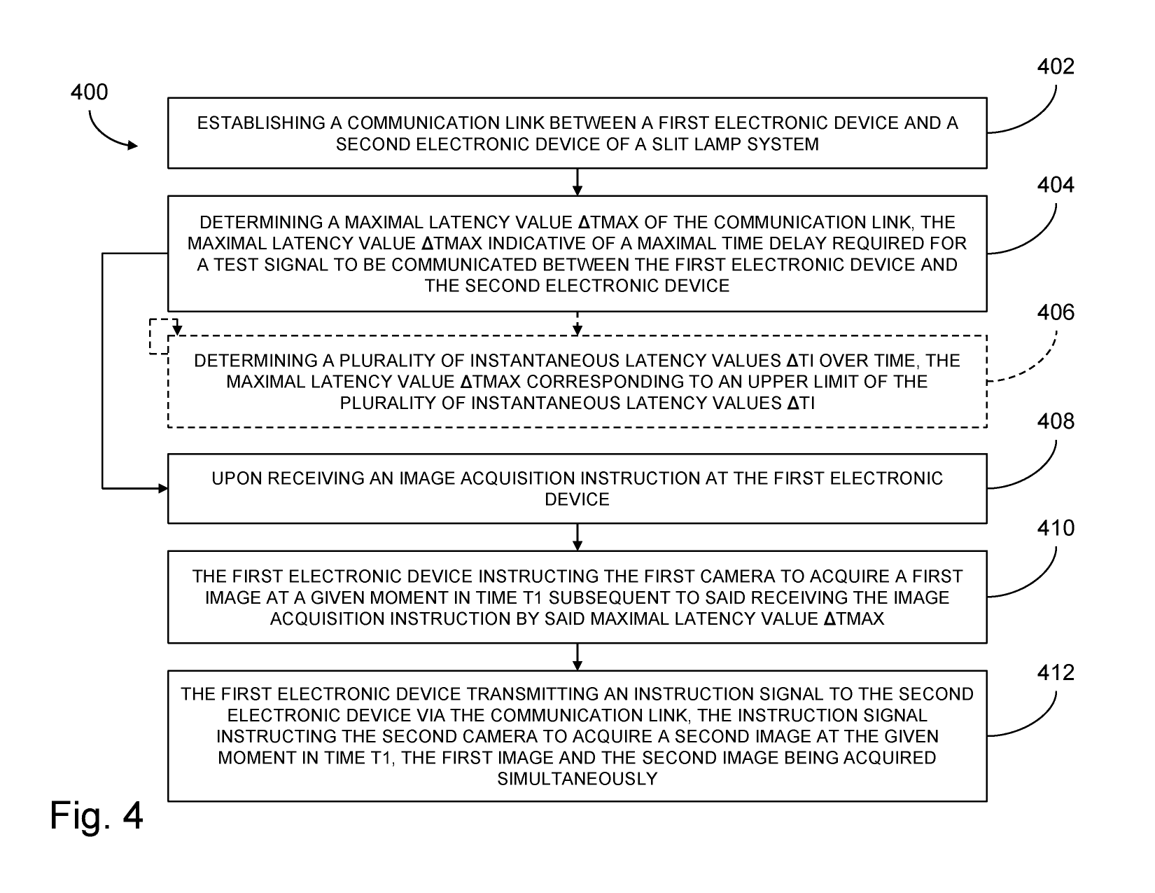

FIG. 4 shows a flow chart of an example of a method 400 of synchronizing image acquisition using a slit lamp system such as the one shown and described with reference to FIG. 1.

At step 402, a communication link is established between a first electronic device and a second electronic device of the slit lamp system. The communication link can be Bluetooth® communication link (such as Bluetooth Low Energy®), a Wi-Fi communication link, a dedicated communication link, or a combination of both depending on the embodiment.

At step 404, a maximal latency value Δtmax of the communication link is determined. The maximal latency value Δtmax is generally indicative of a maximal time delay required for a test signal (or any other signal) to be communicated between the first electronic device and the second electronic device of the slit lamp system. As will be described below, the maximal latency value Δtmax may be a constant value stored on a memory of the first electronic device or be a variable which has to be measured in real time or quasi-real time.

At optional step 406, instantaneous latency values Δti are measured over time at a regular or irregular frequency. For instance, the instantaneous latency values Δti can be determined once every 500 ms, preferably once every 250 ms, and most preferably once every 100 ms. In embodiments where such instantaneous latency values Δti are measured over time, the maximal latency value Δtmax may correspond to an upper limit of the previously measured instantaneous latency values Δti.

At step 408, an image acquisition instruction is received at the first electronic device at a given moment in time t0. The image acquisition instruction may be received from physical actuator(s) via a controller of the slit lamp system or from graphical element(s) of the first and second electronic devices, depending on the embodiment. In any case, upon receiving the image acquisition instruction at the first electronic device, the first electronic device is configured for performing the two following steps 410 and 412 simultaneously or quasi simultaneously (in accordance with any order thereof).

At step 410, the first electronic device instructs the first camera to acquire a first image at a given moment in time t1 subsequent to the reception of the image acquisition instruction by the maximal latency value Δtmax, i.e., t1 = t0 + Δtmax.

At step 412, the first electronic device transmits an instruction signal to the second electronic device via the communication link. The instruction signal instructs the second camera to acquire a second image at the given moment in time t1. Therefore, when the given moment in time t1 arrives, the first camera acquires the first image while the second camera acquires the second image, resulting in the first image and the second image being acquired simultaneously.

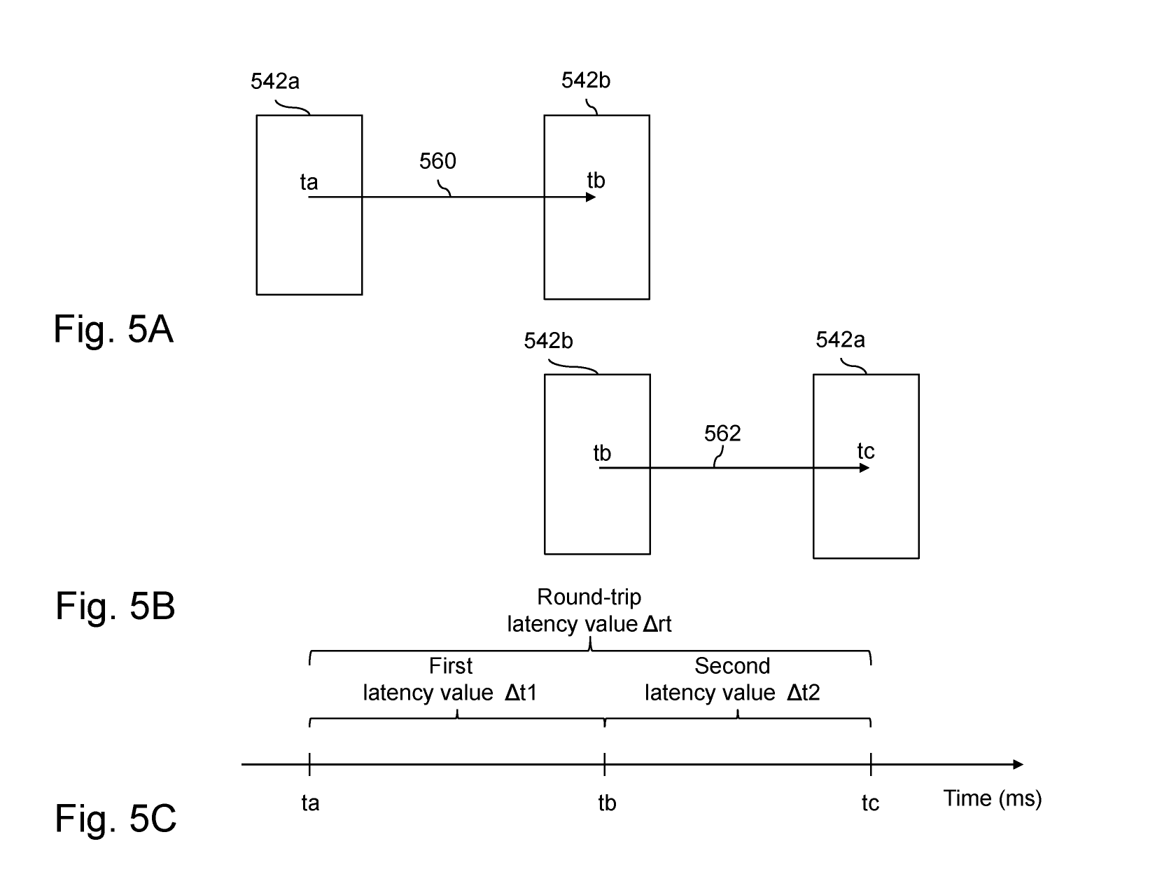

In some embodiments, the calculations of the latency values associated with the communication link established between the first and second electronic devices can differ. For instance, the latency values may be based on one-way latency (e.g., forward latency, return latency) or round-trip latency (including both the forward latency and the return latency). For instance, in some embodiments, a latency value based on a round-trip latency is described with reference to FIGS. 5A-5C. First, at moment in time ta, a test signal 560 is transmitted from a first electronic device 542a to a second electronic device 542b. Then, at moment in time tb, the second electronic device 542b receives the test signal 560 and immediately returns it towards the first electronic device 542a. Ultimately, at moment in time tc, a return 562 of the test signal is received at the first electronic device 542a. When one measures a given time delay extending between the transmission of the test signal and the reception of the return of the test signal (e.g., by subtracting a later timestamp associated with the moment in time tc and a former timestamp associated with the moment in time ta), and divides the given time delay by two, an instantaneous latency value Δti can be determined. In this example, the instantaneous latency value Δti corresponds to an average between a first latency value Δt1 required for the test signal 560 to be communicated from the first electronic device 542a to the second electronic device 542b and a second latency value Δt2 required for the return 562 of the test signal to reach the first electronic device 542a.

In some other embodiments, the instantaneous latency value Δti can be determined based on one-way latency values. For instance, by measuring either one or both of first and second latency values Δt1 and Δt2. The first latency value Δt1 may be determined as follows. First, at moment in time ta, the first electronic device transmits a test signal to the second electronic device. Then, the second electronic device receives the test signal from the first electronic device at moment in time tb. Then, the first latency value Δt1 can be determined by measuring a time delay between a first timestamp associated with the moment in time ta and a second timestamp associated with the moment in time tb. It is intended that the test signal used in these latency determination techniques may be selected to have a size (e.g., 100 ko) corresponding to the instruction signal which is to be communicated between the first and second electronic devices during the execution of the method of synchronizing image acquisition.

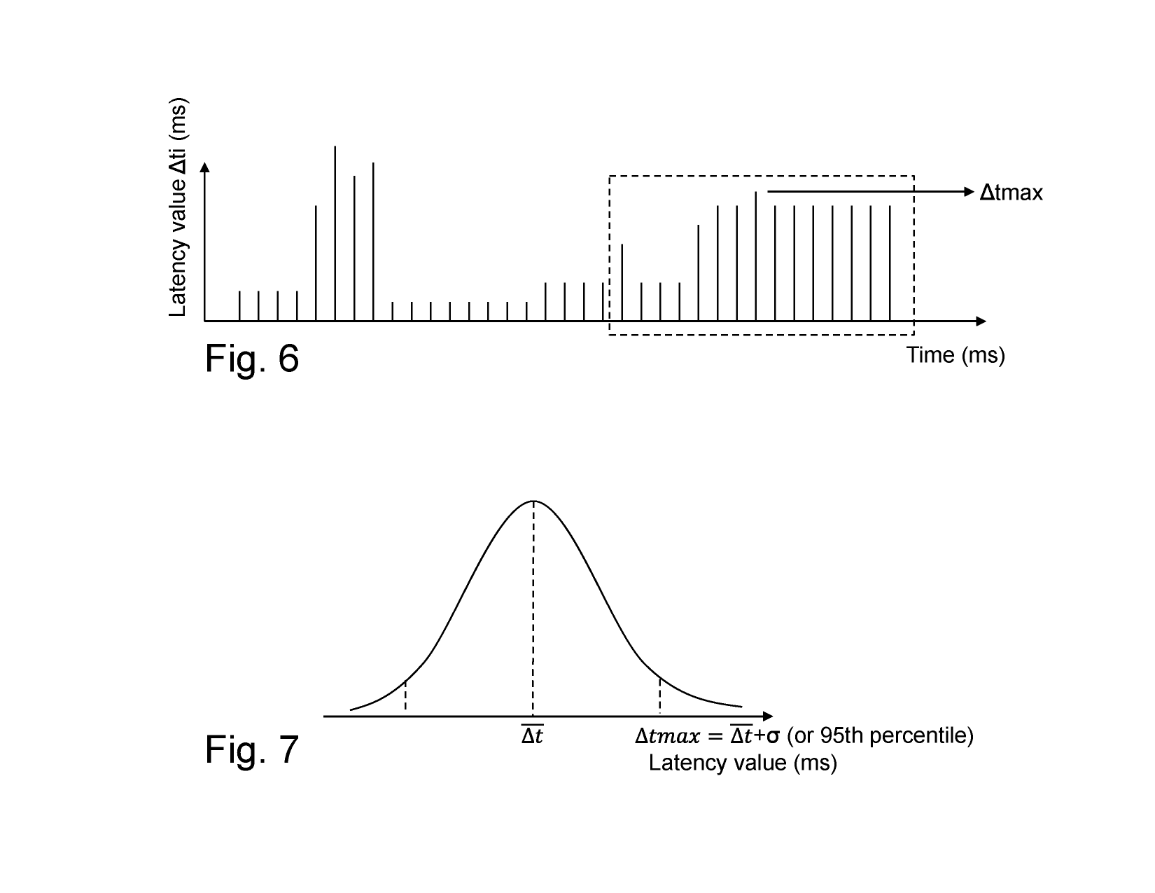

It is intended that the maximal latency value Δtmax discussed above can correspond to an upper limit of some or all of the instantaneous latency values Δti. In some embodiments, the maximal latency value Δtmax is determined based on a number of immediately preceding latency values Δti that are continuously measured over time. These immediately preceding latency values Δti are emphasized by the dashed rectangular area of FIG. 6. For instance, the immediately preceding latency values Δti on which the maximal latency value Δtmax is based may include at most 20 immediately preceding latency values Δti, preferably at most 10 immediately preceding latency values Δti, and most preferably at most 5 immediately preceding latency values Δti.

In some embodiments, a statistical distribution of the instantaneous latency values Δti is determined. In these embodiments, the maximal latency value Δtmax can correspond to a median of the statistical distribution plus one variance value σ of the statistical distribution such as shown in FIG. 7. In some other embodiments, two or more variance values may be used. In some preferred embodiment, the maximal latency value Δtmax can correspond to a 90^{th} percentile, 95^{th} percentile and/or a 98^{th} percentile of the statistical distribution. The statistical distribution can be a normal distribution, a Student distribution or any other suitable statistical distributions reflecting real world scenarios.

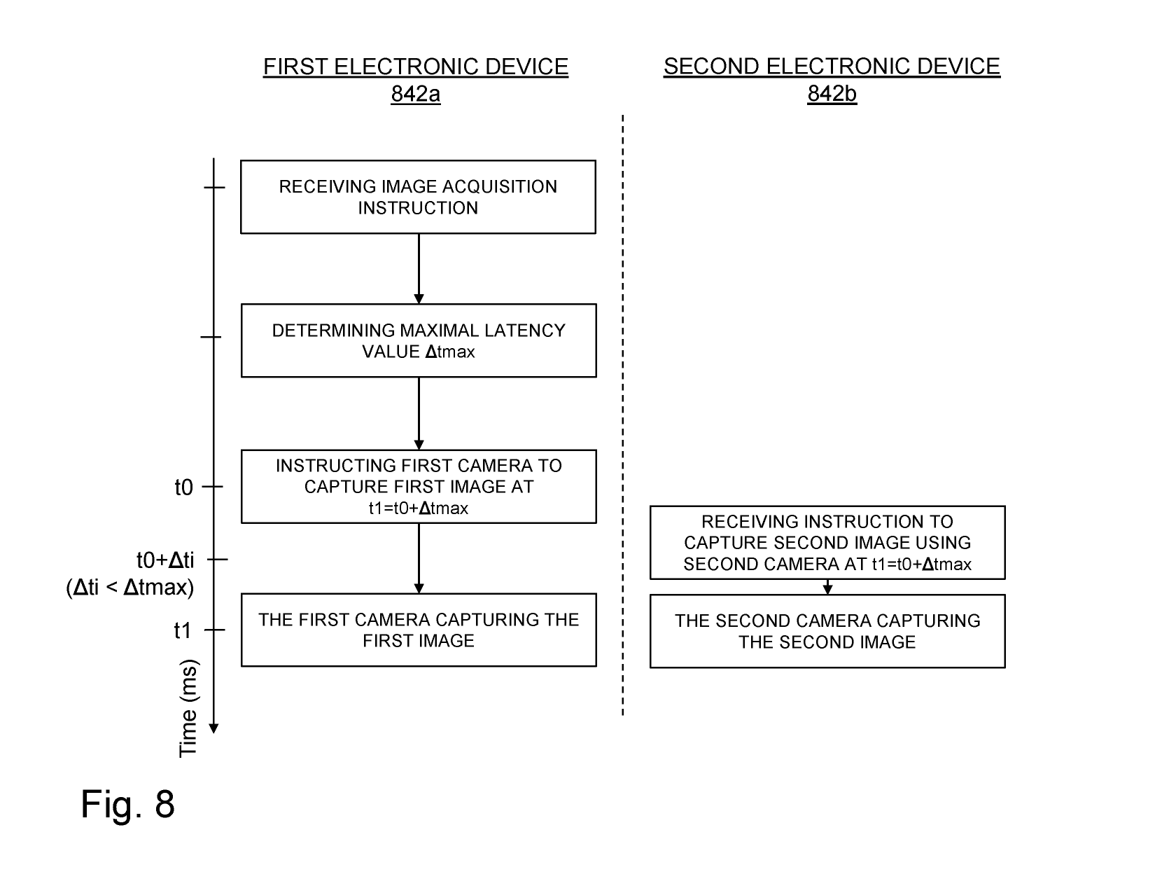

FIG. 8 shows an example sequence of events occurring when an example iteration of the method disclosed herein is performed. This sequence of events is meant to be exemplary only. As depicted, an image acquisition instruction is received at a first electronic device 842a. The image acquisition instruction may be received via a communication link established between the first electronic device 842a and the controller of the slit lamp system or any other controller. Then, a maximal latency value Δtmax is determined. This determination can include a step of retrieving the maximal latency value Δtmax from a computer-readable memory or a step of calculating the most recent maximal latency value Δtmax associated with the communication link. Then, at moment in time t0, the first electronic device 842a instructs the first camera to capture a first image at moment in time t1 which corresponds to the first moment in time t0 plus the previously determined maximal latency value Δtmax. Simultaneously or quasi simultaneously, the first electronic device 842a transmits an instruction signal to the second electronic device 842b. This instruction signal carries instructions to the second camera to capture a second image at the moment in time t1, which also corresponds to the first moment in time t0 plus the previously determined maximal latency value Δtmax introduced a few lines ago. In some embodiments, the reception of the instruction signal at the second electronic device 842b is so closely matching with the moment in time t1 that the instruction to capture the second image is sent immediately upon reception of the instruction signal. In some other embodiments, the first and second electronic devices 842a and 842b have to perform a waiting step prior to actually transmitting the instruction to capture the images to the first and second cameras, thereby ensuring that both the first and second images are acquired at the desired moment in time t1.

As discussed above, the definition of the maximal latency value Δtmax can differ as it can be measured or determined in different manners. However, it is meant that the maximal latency value Δtmax can correspond to a time value which is as small as possible without risking the first image being captured by the first camera before the second electronic device 842b has had a chance to receive the instruction signal from the first electronic device 842a, in which case synchronization of the image capture would not be possible. In some embodiments, the maximal latency value Δtmax is measured once and used as a constant value for each subsequent iteration of the method. However, in some other embodiments, especially those where traffic on the communication link may vary, the maximal latency value Δtmax is monitored in real time to ensure that it matches current network conditions as much as possible, thereby capturing the first and second images as fast as possible, which can be convenient when imaging a living (and potentially moving) subject.

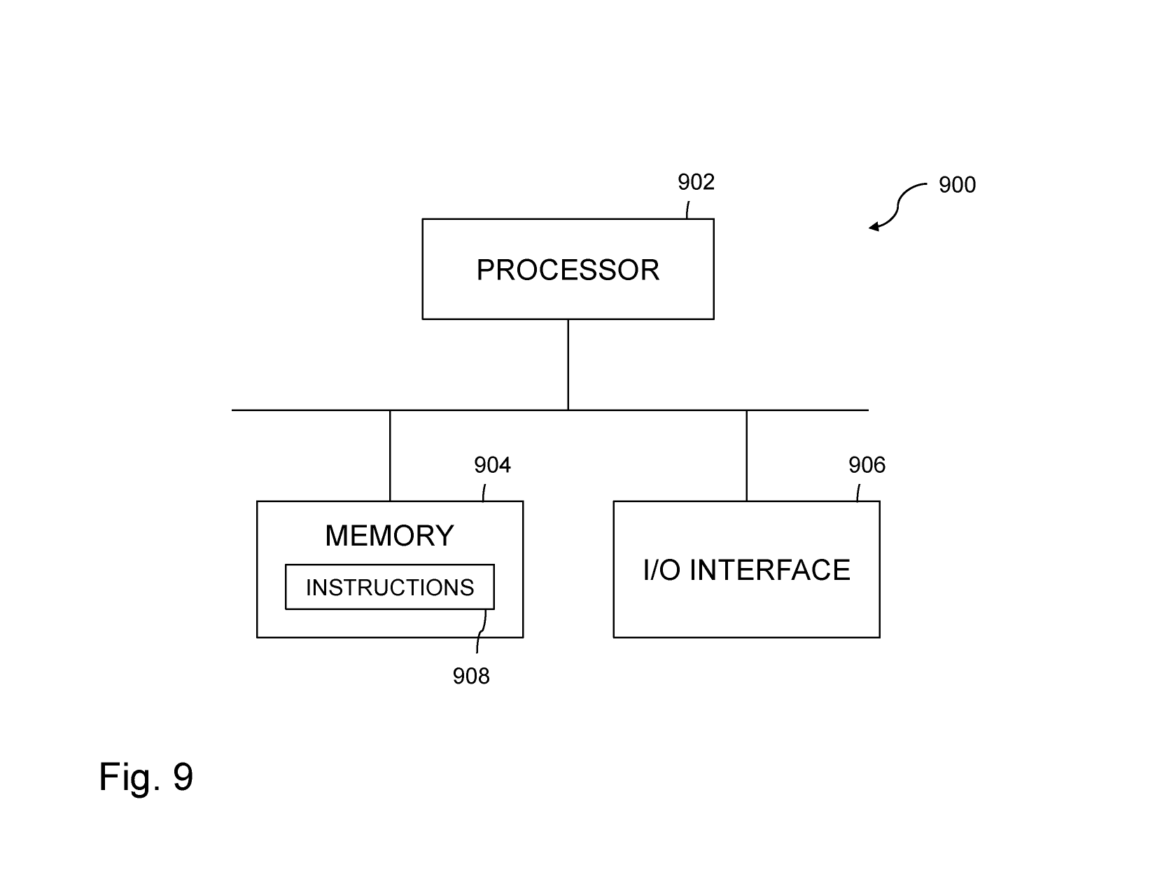

Referring now to FIG. 9, the controller can be provided as a combination of hardware and software components. The hardware components can be implemented in the form of a computing device 900, an example of which is described with reference to FIG. 9. The computing device 900 can have a processor 902, a memory 904, and I/O interface 906. Instructions 908 for synchronizing image acquisition can be stored on the memory 904 and accessible by the processor 902.

The processor 902 can be, for example, a general-purpose microprocessor or microcontroller, a digital signal processing (DSP) processor, an integrated circuit, a field-programmable gate array (FPGA), a reconfigurable processor, a programmable read-only memory (PROM), a programmable logic controller (PLC), or any combination thereof.

The memory 904 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 906 enables the computing device 900 to interconnect with one or more input devices, such as mouse(s), keyboard(s), sensor(s), and/or actuator(s), or with one or more output devices such as display screen(s), accessible memory system(s), and/or external networks.

Each I/O interface 906 enables the controller to communicate with other components, to exchange data with other components, to access and connect to network resources, to server applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fibre optics, satellite, mobile, wireless (e.g. Wi-Fi, WiMAX), SS7 signalling network, fixed line, local area network, wide area network, and others, including any combination of these.

The computing device 900 and any software application that can be ran by the computing device 900 are meant to be examples only. Other suitable embodiments of the controller can also be provided, as it will be apparent to the skilled reader.

In some embodiments, network conditions of the communication link may change suddenly, which may underestimate the actual maximal latency value Δtmax of the communication link. In these instances, the method may not yield synchronized image acquisition. In these instances, the system can be provided with a synchronization confirmation module which can confirm, either via image processing or timestamp comparison, whether the first and second images were acquired simultaneously, and if not, if they are candidates for a satisfactory stereoscopic image. The synchronization confirmation module may be run by the controller of the slit lamp system, by an internal controller of the first electronic deice, by an internal controller of the second electronic device, or a combination thereof. The synchronization confirmation module may involve conventional computer vision to confirm whether asynchronization has occurred in some embodiments. However, in some other embodiments, the synchronization confirmation module may execute a machine learning algorithm which may be compared the first and second images to confirm whether asynchronization has occurred. In cases where asynchronization is determined to have occurred, the synchronization confirmation module may repeat the steps of the method disclosed herein to try to acquire first and second simultaneous images as rapidly as possible.

As can be understood, the examples described above and illustrated are intended to be exemplary only. For example, although the disclosure above describes the use of the method of synchronizing image acquisition in the context of a slit lamp system, it is intended that the method of synchronizing image acquisition can be performed in other contexts. Any one of the first and second electronic devices may perform the method of synchronizing image acquisition described herein. Accordingly, the image acquisition instruction may be sent at either the first electronic device or the second electronic device. As such, the first electronic device may be interchanged with the second electronic device, or vice versa, depending on the embodiment. It is intended that the controllers of the first and second electronic devices have internal clocks which are synchronized with one another. Accordingly, when the first and second electronic devices execute an action at a moment in time x (i.e., timestamp x), the action is performed simultaneously on both electronic devices. In some embodiments, the internal clocks of the controllers of the first and second electronic devices are not synchronized to one another. In these embodiments, a clock delay may be calculated and taken into consideration in the determination of the moment in time at which the first and second images are to be acquired. For instance, in some other embodiments, the acquisition of the first image and the acquisition of the second image can be temporally by one another by any time given time value, which may or may not be dependent on the maximal latency value of the communication link. As such, in certain embodiments such as those described above, this given time value can correspond to maximal latency value Δtmax, however it is not necessary in all embodiments of the present disclosure. The scope is indicated by the appended claims.

What is claimed is:

1. A slit lamp system comprising:
a slit lamp apparatus having a patient area;
a first electronic device having a first camera facing the patient area,
a second electronic device having a second camera facing the patient area, the first and second electronic devices communicating with one another via a communication link having a maximal latency value Δtmax; and
an image acquisition actuator communicatively coupled to the first electronic device and transmitting an image acquisition instruction to the first electronic device upon actuation;
wherein, upon receiving the image acquisition instruction, the first electronic device performs the steps of:
instructing the first camera to acquire a first image at a given moment in time t1 subsequent to said receiving the image acquisition instruction by the maximal latency value Δtmax; and
transmitting a given instruction signal to the second electronic device via the communication link, the given instruction signal instructing the second camera to acquire a second image at the given moment in time t1, resulting in the first image and the second image being acquired simultaneously.

2. The slit lamp system of claim 1 wherein the first electronic device is configured for determining the maximal latency value Δtmax, said determining the maximal latency value Δtmax including: i) the first electronic device transmitting a test signal to the second electronic device, ii) the first electronic device receiving a return of the test signal from the second electronic device, iii) measuring a time delay between said transmitting the test signal and said receiving the return, and iv) dividing the time delay by two to yield an instantaneous latency value Δti.

3. The slit lamp system of claim 1 wherein the first and second electronic devices are configured for determining the maximal latency value Δtmax, said determining the maximal latency value Δtmax including: i) the first electronic device transmitting a test signal to the second electronic device at a first timestamp, ii) the second electronic device receiving the test signal from the first electronic device at a second timestamp, iii) measuring a time delay between the second timestamp and the first timestamp, and associating the time delay to an instantaneous latency value Δti.

4. The slit lamp system of claim 2 or 3 further comprising determining a plurality of instantaneous latency values Δti over time, the maximal latency value Δtmax corresponding to an upper limit of the plurality of instantaneous latency values Δti.

5. The slit lamp system of claim 4 wherein said determining the plurality of instantaneous latency values over time is performed once every 500 ms, and preferably once every 250 ms.

6. The slit lamp system of claim 4 or 5 wherein the maximal latency value Δtmax corresponds to a maximum of a number of immediately preceding latency values Δti, the number of immediately preceding latency values Δti including at most 20 immediately preceding latency values Δti, preferably at most 10 immediately preceding latency values Δti, and most preferably at most 5 immediately preceding latency values Δti.

7. The slit lamp system of any one of claim 1 to 6 wherein the first electronic device is one of: a smart phone, an electronic table, and a digital single-lens reflex (DSLR) camera system.

8. The slit lamp system of any one of claim 1 to 7 wherein the image acquisition actuator is a physical actuator.

9. The slit lamp system of any one of claim 1 to 8 wherein the image acquisition actuator is a graphical element of a graphical user interface.

10. The slit lamp system of claim 9 wherein the graphical user interface is part of any one of the first and second electronic devices.

11. The slit lamp system of any one of claim 1 to 10 wherein the slit lamp apparatus has a mounting bracket mounted thereto, the mounting bracket receiving the first and second electronic devices.

12. A method of synchronizing image acquisition using a first electronic device and a second electronic device, the first electronic device having a first camera, the second electronic device having a second camera, the method comprising:
establishing a communication link between the first electronic device and the second electronic device;
determining a maximal latency value Δtmax of the communication link, the maximal latency value Δtmax indicative of a maximal time delay required for a test signal to be communicated between the first electronic device and the second electronic device;
upon receiving an image acquisition instruction at the first electronic device,
the first electronic device instructing the first camera to acquire a first image at a given moment in time t1 subsequent to said receiving the image acquisition instruction by said maximal latency value Δtmax; and
the first electronic device transmitting an instruction signal to the second electronic device via the communication link, the instruction signal instructing the second camera to acquire a second image at the given moment in time t1, resulting in the first image and the second image being acquired simultaneously.

13. The method of claim 12 wherein said determining the maximal latency value Δtmax includes: i) transmitting the test signal to the second electronic device, ii) receiving a return of the test signal from the second electronic device, iii) measuring a given time delay between said transmitting the test signal and said receiving the return, and iv) dividing the time delay by two to yield an instantaneous latency value Δti.

14. The method of claim 12 wherein determining the maximal latency value Δtmax includes: i) the first electronic device transmitting the test signal to the second electronic device at a first timestamp, ii) the second electronic device receiving the test signal from the first electronic device at a second timestamp, iii) measuring a given time delay between the second timestamp and the first timestamp, and associating the time delay to an instantaneous latency value Δti.

15. The method of claim 13 or 14 further comprising determining a plurality of instantaneous latency values Δti over time, the maximal latency value Δtmax corresponding to an upper limit of the plurality of instantaneous latency values Δti.

16. The method of claim 15 wherein said determining the plurality of instantaneous latency values Δti is performed once every 500 ms, and preferably once every 250 ms..

17. The method of claim 15 or 16 wherein the maximal latency value Δtmax corresponds to a maximum of a number of immediately preceding latency values Δti, the number of immediately preceding latency values Δti including at most 20 immediately preceding latency values Δti, preferably at most 10 immediately preceding latency values Δti, and most preferably at most 5 immediately preceding latency values Δti.

18. The method of any one of claim 15 to 17 further comprising determining a statistical distribution of the plurality of instantaneous latency values Δti, wherein the maximal latency value Δtmax corresponding to a median of the statistical distribution plus a variance value σ of the statistical distribution.

19. The method of any one of claim 15 to 17 further comprising determining a statistical distribution of the plurality of instantaneous latency values Δti, the maximal latency value Δtmax corresponding to a 95^{th} percentile of the statistical distribution.

20. The method of claim 12 wherein the communication link is one of a Bluetooth® communication link, a Wi-Fi communication link, and a dedicated communication link.
